(19) United States
(12) Patent Application Publication
(10) Pub. No.: US 2003/0115623 A1
(43) Pub. Date: Jun. 19, 2003

(54) Methods and compositions for producing donor cells, as well as tissues and organs comprising the same

(76) Inventor: Hal Sternberg, Berkeley, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/286,401

(22) Filed: Nov. 1, 2002

Related U.S. Application Data
(63) Continuation-in-part of application No. PCT/US01/13669, filed on Apr. 27, 2001.
(60) Provisional application No. 60/202,377, filed on May 4, 2000.

(51) Int. Cl. A61K 48/00

(52) U.S. Cl. 800/21; 424/93.21

(57) ABSTRACT

Methods and compositions are provided for producing at least one donor cell that has a genome identical to that of a host. In the subject methods, an embryo is first produced by combining a nucleus obtained for a cell of said host with a recipient cell, e.g. an enucleated oocyte. The resultant embryo is then matured under conditions sufficient to produce a non-sentient organism that includes the donor cell. Finally, the donor cell is harvested from the non-sentient organism for subsequent use. Also provided are kits for use in practicing the subject invention. The subject methods find use in a variety of different applications, including tissue and organ transplant applications.

### CROSS-REFERENCE TO RELATED APPLICATIONS

This application is a continuation-in-part of PCT Application Serial No. PCT/US01/13669 filed on Apr. 27, 2001 and designating the U.S.; which application claims priority under 35 U.S.C. § 119 (e) to the filing date of the U.S. Provisional Application Serial No. 60/202,377 filed on May 4, 2000; the disclosures of which applications are herein incorporated by reference.

### INTRODUCTION

1. Field of the Invention

The field of this invention is transplantation.

2. Background of the Invention

Transplantation is the transfer of a tissue or an organ, where the transfer may be: (a) between two individuals of the same species (i.e. transplant of an allograft or allotransplantation); (b) from one species to another (i.e. transplant of a xenograft or xenotransplantation); or from one site to another in the same individual (i.e. transplant of a autograft or autotransplantation). In people, transplantation has revolutionized medicine by providing treatment options for otherwise non-treatable conditions. Tissues and organs that have been transplanted include: kidneys, corneas, tendons, nerves, bones, skin, hearts, bone marrow, lungs, fallopian tubes, pancreas, liver and the like.

One of the technical hurdles facing transplantation is graft rejection. Despite efforts to avoid graft rejection through host-donor tissue type matching, in the majority of transplantation procedures where a donor organ is introduced into a host, chronic immunosuppressive therapy following transplantation is critical to the maintained viability of the donor organ in the host. A variety of immunosuppressive agents have been developed for use in chronic immunosuppression, including: azathioprine, methotrexate, cyclophosphamide, FK-506, rapamycin, corticosteroids and cyclosporin.

Another obstacle to transplantation is that demand for organs far exceeds supply. At any given moment, the number of individuals waiting to receive transplant organs is much greater than the number of organ donors available. As such, there is a chronic shortage of donor organs. While xenotransplantation has the potential to provide at least partial solution to this problem, it has yet not progressed out of the research stage.

As such, there is a continued need for the development of new transplant methodologies that overcome one or more of the above hurdles. Of particular interest would be the development of a methodology that could overcome both the problem of graft rejection and the problem of limited donor organ supply.

Relevant Literature

Of interest are: WO 97/07668 and WO 97/07669. Also of interest are: Campbell et al., Theriogenology (1996) 45:287; Trounson et al., Reprod. Fertil. Dev. (1998) 10:645-650; Wakayama et al., Semin. Cell Dev. Biol (1999) 10:253-258; Wolf et al., Biol. Reprod. (1999) 60:199-204; Cibelli et all, Science (1998) 280:1256-1258; Wilmut et al., Nature (1997) 385:810-813; Campbell et al., Nature (1996) 380:64-66; Prather et al., J. Reprod. Fertil. Suppl. (1990) 41:125-134; and Wells et al., Biol. Reprod. (1997) 57:385-393.

### SUMMARY OF THE INVENTION

Methods and compositions are provided for producing at least one donor cell that has a genome identical to that of a host. In the subject methods, an embryo is first produced by combining a nucleus obtained from a cell of said host with a recipient cell, e.g. an enucleated oocyte. The resultant embryo is then matured under conditions sufficient to produce a non-sentient organism that includes the donor cell. Finally, the donor cell is harvested from the non-sentient organism for subsequent use. Also provided are kits for use in practicing the subject invention. The subject methods find use in a variety of different applications, including tissue and organ transplant applications.

### DESCRIPTION OF THE SPECIFIC EMBODIMENTS

Methods and compositions are provided for producing at least one donor cell that has a genome identical to that of a host. In the subject methods, an embryo is first produced by combining a nucleus obtained for a cell of said host with a recipient cell, e.g. an enucleated oocyte. The resultant embryo is then matured under conditions sufficient to produce a non-sentient organism that includes the donor cell. Finally, the donor cell is harvested from the non-sentient organism for subsequent use. Also provided are kits for use in practicing the subject invention. The subject methods find use in a variety of different applications, including tissue and organ transplant applications. In further describing the subject invention, the methods-will be discussed first in greater detail followed by a review of representative applications in which the subject methodology finds use.

Before the, subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

Methods

As summarized above, the subject methods are methods of producing at least one donor cell, as well as a tissue or organ that includes the cell, that has a genome identical to that of a host. By having a genome identical to that of a host is meant that the genome of the cell produced by the subject methods has a genome substantially similar to, if not identical to, that of the host cell from which it is derived such that the cell produced by the subject methods is considered to be a clone of the host cell from which it is derived. In other words, the host cell and the cells produced by the subject method are identical in the same sense that the cells produced by the methods described in WO 96/07732; WO 97/07669 and WO 97/07668 are identical with the donor cells from which they are derived. The first step of the subject methods is to produce an embryo that has a genome of interest, i.e. a genome identical to that of the subject with which the donor cell, tissue or organ thereof produced by the subject methods is to be employed. The embryo may be produced using any convenient technology.

In many embodiments of the subject methods, the embryo is produced by combining a nucleus from the host with a suitable recipient cell under conditions sufficient to produce an embryo capable of maturation under embryo maturation conditions. The nucleus that is employed is generally one that has been harvested from a quiescent cell. A variety of quiescent cells may be employed as the source of the nucleus. In many embodiments, the quiescent cell is a cell of the subject with which the ultimately produced donor cell is to be employed. Any convenient cell that is quiescent or may be induced to enter quiescence (i.e. the G₀ stage) may be employed, where suitable cells are cells of normal karyotype and include embryonic, fetal and adult somatic cells. The quiescent cell that serves as the nucleus source may or may not be transgenic, i.e. it may or may not have a genome that has been manipulated via recombinant DNA protocols, e.g. through insertion of an exogenous gene, through knockout of an endogenous gene, etc.

The quiescent cell from which the nucleus is obtained is, in many embodiments, a cell derived from the subject with which the donor cell produced by the subject methods is to be employed, i.e. the host and ultimate subject are the same organisms. The host is generally an animal, where the animal may be bird, amphibian, fish, etc., and is often a mammal. Mammals of interest include: placental animals, e.g. ungulates (such as sheeps, cattle, goats, water buffalo, camels, pigs, and the like), horses, llamas, rodents, e.g. rats, mice, etc., rabbits, primates, e.g. monkeys, baboons, gorillas, humans, etc.

As mentioned above, the diploid nucleus of the host quiescent cell is combined with a suitable recipient cell. Any convenient recipient cell may be employed, where suitable recipient cells include, but are not limited to: metaphase I to metaphase II oocytes, zygotes, two cell embryos, etc. Generally the recipient cell is an enuclated cell. Methods for enucleating cells are known in the art, including cell splitting as described in Willadsen, Nature (1986) 320:63-65; aspiration of the first polar body and neighboring cytoplasm, as described in Smith & Wilmut, Biol. Reprod. (1989) 40:1027-1035; enuclation with DNA specific fluorochrome as described in Tsunoda et al., J. Reprod. Fertil. (1988) 82:173; non invasive enucleation protocols, such as ultraviolet irradiation; and the like. In many embodiments, the recipient cell is an enucleated metaphase II oocyte, an enucleated unactivated oocyte or an enucleated preactivated oocyte. Specific recipient cells of interest include: the “MAGIC recipient” as described in WO 97/07668, the disclosure of which is herein incorporated by reference; the “GOAT” recipient, as described in Campbell et al., Biol. Reprod. (1993) 49:933-942, and the “Universal Recipient,” as described in Campbell et al., Biol. Reprod. (1994) 50:1385-1393.

The nucleus of the host cell may be combined with the recipient cell to produce the embryo using any convenient protocol. Representative technologies that may be employed include: exposure of the cells to fusion promoting agents, e.g. polyethylene glycol; the use of an inactivated virus, e.g. Sendai virus, to promote fusion; the use of electrical stimulation; microinjection; and the like.

At some point during the above procedure or immediately after transfer of the nucleus to the recipient, the recipient cell is activated, i.e. stimulated by parthenogenetic activation, in many embodiments of the invention. Although any convenient activation protocol may be employed, in many embodiments the recipient cell is generally subjected to one or more electrical pulses following transfer of the diploid nucleus, where the strength of the pulse and periodicity where a plurality of pulses are employed may vary depending on the particular recipient cell.

The above steps result in the production of an embryo that is then capable of maturation into an adult organism under suitable conditions. The above steps are further described in WO 96/07732; WO 97/07669 and WO 97/07668; the disclosures of which are herein incorporated by reference.

Following embryo production, the resultant embryo is then matured under conditions sufficient to produce a non-sentient organism that includes the donor cell, tissue or organ thereof. The embryo may be matured using any convenient means, including both in vivo and in vitro means. In vivo means that may be employed include in utero means, in which the embryo is implanted into a surrogate mother that includes a uterus in which the embryo is capable of maturing. Procedures for implanting an embryo into a uterus (i.e. for performing embryo transfer) are known in the art, and include those described in U.S. Pat. Nos: 6,027,443; 5,916,144; 5,904,665; 5,656,010; 5,558,636; 5,536,243; 5,147,299; and 4,474,576, as well as WO 97/14365; the disclosures of which are herein incorporated by reference. See also: Baba et al., Fertil Steril. (2000) 73(1):123-5; Goudas et al., Fertil Steril. (1998)70(5):878-82; Krampl et al., Fertil Steril. (1995) 63(2):366-70; Ho et al., J Formos Med Assoc. (1992) 91(7):708-11; Walters, J Am Vet Med Assoc. (1984) 184(6):649; Balmaceda et al., Fertil Steril. (1992) 57(2):362-5; Kan et al., Hum Reprod. (1999) 14(5):1259-61; Khan et al., Fertil Steril. (1991) 56(1):98-101; and Letterie et al., Fertil Steril. August 1999;72(2):266-8. In vitro maturation means of interest include artificial uterus means, including those described in U.S. Pat. No. 5,218,958, the disclosure of which is herein incorporated by reference.

A feature of the subject invention is that the embryo is matured under conditions that do not give rise to a sentient or conscious animal, i.e. under conditions that produce a non-sentient animal. Any convenient methodology for ensuring that the embryo does not mature into a sentient animal may be employed. In many embodiments, the embryo will be matured under conditions that prevent the formation of a functional forebrain in the animal, where the forebrain is generally considered to be the location of sentience or consciousness. Functional forebrain formation prevention may be accomplished in a number of different ways, including chemical and physical means. As such, the embryo may be contacted with one or more active agents during maturation that prevent formation of a functional forebrain. Examples of such chemical agents include: toxic agents, preferably selectively targeted to forebrain neural cells; agents that selectively turn on inactivating genes present in forebrain neural cells, e.g. agents that turn on apoptosis genes in these cells, where the cells may have been engineered to include a promoter that controls such genes and is selectively turned on following administration of the agent; and the like.

Alternatively, the forebrain may be physically prevented from forming, e.g. by preventing closure of the neural tube during maturation, by removal of one or more forebrain precursor cells or tissues during maturation, etc. Depending on the particular method of maturation employed, this physical intervention is often in utero. In utero surgical protocols are known in the art and described in Olutoye et al., Semin Perinatol. (1999) 23(6):462-73; Simpson et al., JAMA. (1999) 17;282(19):1873-4; Dias, Pediatr Neurosurg. (1999) 30(2):108; Adamsons, N Engl J Med. (1966) 275(4):204-6.; Tulipan et al., Pediatr Neurosurg. (1998) 28(4):177-80; Sutton et al., JAMA (1999) 17;282(19):1826-31; Meuli-Simmen et al., Plast Reconstr Surg. (1995) 96(5):1007-11; Luks, BMJ. (1995)311(7018):1449-50; Harrison; Am J Obstet Gynecol. (1996)174(4):1255-64; and Quintero et al., Lancet. (1995) 23-30;346 Suppl:s18. Preferably, this physical intervention is taken as early as possible during maturation of the embryo into the non-sentient organism. Where desired, the surgically removed cells may be employed for another use, e.g. another medical therapeutic treatment, etc.

The above steps result in the production of a non-sentient organism that includes the donor cell of interest, where the donor cell may or may not be part of a larger collection or aggregation of cells, e.g. in the form of a tissue or organ, etc. Because of the protocol that is employed, the donor cell or cells (e.g. cells of the tissue or organ) have the same genome as the original host from which the nucleus was obtained to produce the non-sentient organism, as described above.

The final step of the subject methods is removal of the cell, tissue or organ from the non-sentient animal for subsequent use. As such, the final step is to harvest the cell, tissue or organ from the non-sentient animal. Any convenient harvesting protocol may be employed, where the particular protocol employed will necessarily depend on the nature of the cell, tissue or organ being harvested. Representative protocols include those described and/or referenced in: Barry, Curr Opin Urol March 1999;9(2):121-7 (kidneys); Wood, Essays Biochem. 1995;29:65-85 (skin); Befeler et al., Transplantation. (1999) 68(9):1423-7 (heart-liver); Bacigalupo et al., Semin Hematol. (2000) 37(1):69-80 (bone marrow); Benfield et al., Chest Surg Clin N Am. (2000) 10(1):189-99 (lung); and Sutherland et al., Transplant Proc. August 1998;30(5):1940-3 (Pancreas).

Utility

The above described methods find use in a variety of different applications. One application of particular interest is in the production of donor cells, tissues and organs for use in transplant procedures. While the above protocols find use in autograft, allograft and xenograft procedures, they are particularly suited for use in autograft or allograft procedures. Autograft procedures in which the subject methods find use are those in which the host from which the nucleus is obtained is the subject with the which the donor cell, tissue or organ thereof is to be employed. Allograft procedures in which the subject methods find use are those in which the host from which the nucleus is obtained is different from the subject with which the donor cell, tissue or organ thereof is to be employed, but is of the same species. Of particular interest is use of the subject methods to in autograft transplant procedures.

The subject methods may be used to produce a variety of different types of donor cells, organs or tissues. Representative cells, organs and tissues that may be produced by using the subject methods include, but are not limited to: kidneys, corneas, tendons, nerves, bones, skin, hearts, bone marrow, lungs, fallopian tubes, pancreas, liver, bowel, stomach, intestine, lymph nodes, ovaries, testes, and the like.

The subject methods may be employed to produce graft cells, tissues and organs for use with a variety of different types of animals and for a variety of different purposes, where representative animals include but are not limited to: placental animals, e.g. ungulates (such as sheeps, cattle, goats, water buffalo, camels, pigs, and the like), horses, llamas, rodents, e.g. rats, mice, etc., rabbits, primates, e.g. monkeys, baboons, gorillas, humans, etc. The cells, tissues or organs may be employed in a number of different applications, where representative applications include, but are not limited to: replacement of poorly or non-functioning organs or tissues, cosmetic applications, e.g. replacement of damaged or scarred skin, growth of hair, etc., and the like.

The following examples are offered by way of illustration and not by way of limitation.

### EXPERIMENTAL

I Embryo Production

A. Ovine Nuclear Transfer

Superstimulation of Donor Ewes and Recovery of Oocytes

Scottish Blackface ewes are synchronized with progestagen sponges for 14 days (Veramix^{J}, Upjohn, UK) and induced to superovulate with single injections of 3.0 mg/day (total 6.0 mg) ovine follicle-stimulating hormone (FSH) (Ovagen^{J}, Immuno-chemical Products Ltd, New Zealand) on two successive days. Ova ulation is induced with as 8 mg single dose of a gonadotropin-releasing hormone analogue (GnRH Receptal^{J}, Hoechst, UK) 24 hours after the second injection of FSH.

Unfertilized metaphase II oocytes are recovered by flushing from the oviduct at 24-29 hours after GnRH injection using Dulbecco's phosphate buffered saline containing 1.0% fetal calf serum (FCS) maintained at 37° C. until use.

Oocyte Manipulation

Recovered oocytes are maintained at 37° C., washed in PBS 1.0% FCS and transferred to calcium free M2 medium containing 10% Fetal Calf Serum (FCS), at 37° C. To remove the chromosomes, (enucleation) oocytes are placed in calcium free M2 containing 10% FCS, 7.5 μg/ml cytochalasin B (Sigma) and 5.0 μg/ml Hoechst 33342 (Sigma) at 37° for 20 minutes. A small amount of cytoplasm from directly beneath the 1st polar body is then aspirated using a 20 μM glass pipette. Enucleation is confirmed by exposing the aspirated portion of cytoplasm to UV light and checking for the presence of a metaphase plate.

Embryo Reconstruction

Groups of 10-20 oocytes are enucleated and placed into 20 μl drops of calcium free M2 medium at 37° C. 5% CO₂ under mineral oil (SIGMA). Each of the following three protocols (a), (b) and (c) are used for embryo reconstruction with a cell from a host sheep.

(a) “MAGIC” (Metaphase Arrested G1/G0 Accepting Cytoplast)

As soon as possible after enucleation, a single cell obtained from a host sheep is placed into contact with the oocyte by using a glass pipette to transfer the cell through the hole previously made in the zona pellucida. The cytoplast/cell couplet is then transferred into the fusion chamber in 200 μl of 0.3M mannitol in distilled water and manually aligned between the electrodes. An AC pulse of 5V was applied for 3 seconds followed by 3 DC pulses of 1.25 kV/cm for 80 μsecs. The couplets are then washed in calcium free M2, 10% FCS at 37° C. and incubated in the same medium under oil at 37° C. 5% CO₂. 30 minutes prior to activation the couplets are transferred to calcium free M2 medium 10% FCS containing 5 μM nocodazole. Activation is induced at 32-34 hours post hCG injection as described below. Following activation the reconstructed zygotes are incubated in medium TC199 (Gibco) 10% FCSW at 37° C. 5% CO₂ for a further 3 hours. They are then washed 3 times for 5 minutes at 37° C. in the same medium without nocodazole and cultured for a further 12-15 hours prior to transfer to temporary recipient ewes.

(b) “GOAT” (G0/G1 Activation and Transfer)

At 32-34 hours post hCG injection a single cell from a host sheep is placed into contact with the enucleated oocyte. The couplet is transferred to the fusion chamber (see below) in 200 μl of 0.3M mannitol, 0.1 mM MgSO₄, 0.001 mM CaCl₂ in distilled water. Fusion and activation are induced by application of an AC pulse of 3V for 5 seconds followed by 3D pulses of 1.25 kV/cm for 80 μsecs. Couplets are then washed in TC199 10% FCS containing 7.5 μ/ml cytochalasin B and incubated in this medium for 1 hour at 37° C. 5% CO₂. Couplets are then washed in TC199 10% FCS at 37° C. 5% CO₂.

(c) Universal Recipient”

Enucleated oocytes are activated (as described below) 32-34 hours post hCG injection and then cultured in TC1199 10% FCS at 37° C. 5% CO₂ for 4-6 hours. A single cell from a host sheep is then placed into contact with the oocyte and fusion induced as described below. The couplets are then incubated in TC199 10% FCS 7.5 μg cytochalasin B for 1 hourat 37° C. 5% CO₂. Couplets are then washed and cultured in TC199 10% FCS at 37° C. 5% CO₂ for a further 8-11 hours.

Fusion and Activation

For activation, oocyte are placed between two parallel electrodes in 200 μl of 0.3M mannitol, 0.1 mM MgSO₄, 0.001 mM CaCl₂ in distilled water (Willadesen, Nature 320 63-65 (1986)). Activation is induced by application of 1DC pulse of 1.25 kV/cm for 80 μs. For fusion, manipulated embryos are treated in a similar manner with the addition that the contact surface between the enucleated oocyte and the cell from the host sheep is arranged parallel to the electrodes. Fusion is induced by application of an AC current of 3V for 5 seconds followed by 3 DC pulses of 1.25 kV/cm for 80 μs.

Embryo Culture and Assessment (All Groups)

After the culture period fused couplets are double embedded in 1% and 1.2% agar (DIFCO) in PBS and transferred to the ligated oviduct of unsynchronized ewes. The couplet is embedded in agar to prevent or reduce immune rejection of the embryo by the recipient ewe and to assist in holding the couplet together. After 6 days recipient ewes are sacrificed and the embryos retrieved by flushing from the oviduct using PBS 10% FCS. Embryos are dissected from the agar chips using 2 needles and development assessed by microscopy. All embryos which are developed to the morula/blastocyst stage are transferred as soon as possible to the uterine horn of synchronized final recipient ewes. In vitro techniques may also be suitable in place of a temporary recipient ewe to achieve development of the embryo to the blastocyst stage.

II. Maturation

Roughly 2 to 18 weeks following implantation, a surgical procedure is employed on the fetus in utero to remove the forebrain tissue.

III. Birth of Non-Sentient Animal

The resultant non-sentient fetus is allowed to mature to term. Following birth the fetus is maintained on life support while a kidney is harvested. The harvested kidney has the same genotype as that of the original host sheep.

IV. Implantation of Kidney Into Original Host Sheep.

The harvested kidney is implanted into the host sheep using conventional transplant protocols. The kidney functions normally and no signs of rejection are observed.

It is evident that the subject invention provides a convenient means of producing donor cells, tissues and organs that can be used in autograft transplantation procedures, as well as allograft and xenograft procedures. As such, the subject methods provide an convenient means for overcoming current problems in transplantation medicine, including graft rejection and limited graft supply. As such, the subject invention provides an important contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and, modifications may be made thereto without departing from the spirit or scope of the appended claims.

What is claimed is:

1. A method of producing at least one donor cell having a genome identical to that of a host, said method comprising:
producing an embryo by transferring a nucleus obtained from a cell of said host into a recipient cell;
maturing said embryo under conditions sufficient to produce a non-sentient organism comprising said at least one donor cell; and
harvesting said at least one donor cell from said non-sentient organism;
whereby said at least one donor cell having a genome identical to that of said host is produced.

2. The method according to claim 1, wherein said recipient cell is an oocyte.

3. The method according to claim 1, wherein said oocyte is enucleated.

4. The method according to claim 1, wherein said non-sentient organism lacks a functional forebrain.

5. The method according to claim 4, wherein said maturing comprises preventing formation of said fore brain.

6. The method according to claim 4, wherein said maturing comprises removal of said forebrain or precursors thereof at a time prior to said harvesting.

7. The method according to claim 1, wherein said maturing comprises growing said embryo in a uterus.

8. The method according to claim 1, wherein said method produces a plurality of said donor cells.

9. The method according to claim 8, wherein said plurality of donor cells are in the form of a tissue or organ.

10. The method according to claim 1, wherein said host is mammalian.

11. A method of producing mammalian tissue, said method comprising:
producing an embryo by transferring a nucleus obtained from a cell of host into an enucleated oocyte;
maturing said embryo under conditions sufficient to produce a non-sentient organism comprising said tissue, wherein said non-sentient organism lacks a functional forebrain; and
harvesting said tissue from said non-sentient organism;
whereby said tissue is produced.

12. The method according to claim 11, wherein said tissue is an organ.

13. The method according to claim 11, wherein said host is a primate.

14. The method according to claim 11, wherein said host is an ungulate.

15. The method according to claim 11, wherein said maturing comprises preventing formation of said fore brain.

16. The method according to claim 11, wherein said maturing comprises removal of said forebrain or precursors thereof at a time prior to said harvesting.

17. The method according to claim 11, wherein said maturing comprises growing said embryo in a uterus.

18. A method of transplanting donor mammalian tissue to a mammalian recipient, said method comprising:
(a) producing said donor mammalian tissue by the method comprising:
(i) producing an embryo by transferring a nucleus obtained from a cell of a host into an enucleated oocyte;
(ii) maturing said embryo under conditions sufficient to produce a non-sentient organism comprising said donor mammalian tissue, wherein said non-sentient organism lacks a functional forebrain; and
(iii) harvesting said donor mammalian tissue from said non-sentient organism, whereby said tissue is produced; and
(b) transplanting said harvested donor mammalian tissue to said mammalian recipient;
whereby donor mammalian tissue is transplanted to said mammalian recipient.

19. The method according to claim 18, wherein said host is an ungulate.

20. The method according to claim 18, wherein said host is a primate.

21. The method according to claim 18, wherein said host is said mammalian recipient.

22. The method according to claim 18, wherein said host is not said mammalian recipient.
